(19) United States
(12) Plant Patent Application Publication
(10) Pub. No.: US 2016/0242346 P1
(43) Pub. Date: Aug. 18, 2016

(54) New Guinea Impatiens plant named 'DHARSSCRLT'

(50) ;

(71) Applicant: Gavriel Danziger, Moshav Mishmar Hashiva (IL)

(72) Inventor: Gavriel Danziger, Moshav Mishmar Hashiva (IL)

(21) Appl. No.: 14/544,749

(22) Filed: Feb. 12, 2015

(51) Int. Cl. A01H 5/00

(52) U.S. Cl. PLT/318.7

(57) ABSTRACT

A new and distinct Impatiens cultivar named ‘DHARSSCRLT’ is disclosed, characterized by large scarlet flowers well branched form and good sun resistance. The new variety is an Impatiens, normally produced as an outdoor garden or container plant.

### LATIN NAME OF THE GENUS AND SPECIES

Impatiens hawkeri

### VARIETY DENOMINATION

‘DHARSSCRLT’

### BACKGROUND OF THE INVENTION

The new Impatiens cultivar is the product of a planned breeding program conducted by the inventor, Gavriel Danziger, in Moshav Mishmar Hashiva, Israel. The objective of the breeding program was to produce new Impatiens varieties for ornamental commercial applications. The cross resulting in this new variety was made during February of 2012.

The seed parent is the, unpatented, proprietary variety referred to as Impatiens ‘BM-2884’. The pollen parent is the unpatented, propriety variety referred to as Impatiens ‘BM-1801’. The new variety was discovered in December of 2012 by the inventor in a group of seedlings resulting from the February 2012 crossing, in a research greenhouse in Moshav Mishmar Hashiva, Israel.

Asexual reproduction of the new cultivar was first performed by vegetative cuttings. The cuttings are from an apical bud that has no more than two expanded leaves and three to four immature leaves present. This was first performed at a research greenhouse in Moshav Mishmar Hashiva, Israel in January of 2013 and has shown that the unique features of this cultivar are stable and reproduced true to type in multiple successive generations.

### SUMMARY OF THE INVENTION

The cultivar ‘DHARSSCRLT’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘DHARSSCRLT’ These characteristics in combination distinguish ‘DHARSSCRLT’ as a new and distinct Impatiens cultivar:

1. Large flowers

2. Scarlet flower color

3. Well branched plants

4. Good resistance to sunny conditions

Plants of the new cultivar ‘DHARSSCRLT’ are similar to plants of the seed parent, Impatiens ‘BM-2884’ in most horticultural characteristics, however, plants of the new cultivar ‘DHARSSCRLT’ differ in the following;

1. New variety has scarlet flowers while the parent has magenta flowers.

2. New variety has larger flowers than the parent.

Plants of the new cultivar ‘DHARSSCRLT’ are similar to plants of the pollen parent, Impatiens ‘BM-1801’ in most horticultural characteristics, however, plants of the new cultivar ‘DHARSSCRLT’ differ in the following;

1. The new variety has scarlet flowers while the parent has orange flowers.

2. The new variety is more compact while the parent is more vigorous.

### COMMERCIAL COMPARISON

Plants of the new cultivar ‘DHARSSCRLT’ are comparable to the commercial variety Impatiens ‘DHAR321’ U.S. Plant Pat. No. 25,117. The two Impatiens varieties are similar in most horticultural characteristics; however, the new variety ‘DHARSSCRLT’ differs in the following:

1. Flower color of the new variety is lighter

2. Leaf size of the new variety is larger

3. Growth habit of the new variety is more compact.

Plants of the new cultivar ‘DHARSSCRLT’ can also be compared to the patented commercial variety Impatiens ‘SAKIMP024’, plant patent applied for, application number unknown. The two Impatiens varieties are similar in most horticultural characteristics; however, the new variety ‘DHARSSCRLT’ differs in the following:

1. Flower color of the new variety is lighter and brighter

2. Flower size of the new variety is larger

3. Leaf size of the new variety is larger

### BRIEF DESCRIPTION OF THE PHOTOGRAPHS

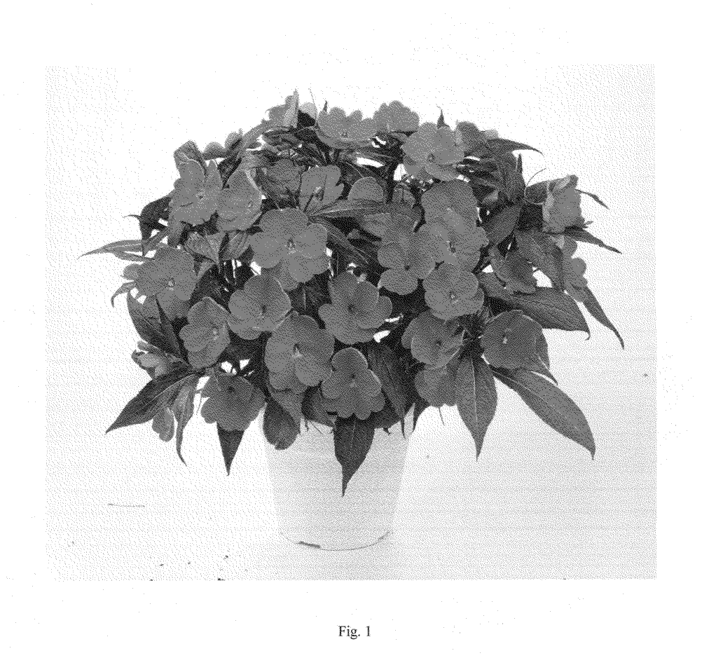

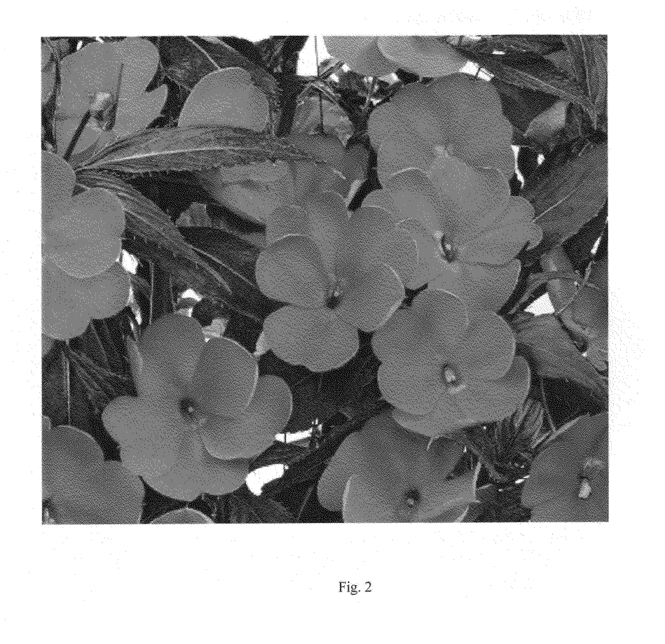

The accompanying photograph in FIG. 1 illustrates in full color a typical plant of ‘DHARSSCRLT’ grown outdoors, in a 16 cm pot. Age of the plant photographed is approximately 14 weeks from a rooted cutting. FIG. 2 illustrates a close up of the flowers The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Mini Colour Chart 2001 except where general terms of ordinary dictionary significance are used. The following observations and measurements describe ‘DHARSSCRLT’ plants grown in the Autumn, in a greenhouse, in Moshav Mishmar Hashiva, Israel. The growing temperature ranged from 20° C. to 30° C. during the day and from 15° C. to 25° C. during the night. General light conditions are bright, normal sunlight. Measurements and numerical values represent averages of typical plant types.
Botanical classification: Impatiens ‘DHARSSCRLT’
Propagation:
   Type of propagation typically used.—Cutting.
   Time to initiate roots.—10-14 days Summer, 14-20 days Winter.
   Root description.—Fibrous adventitious roots.
Plant:
   Age of plant described.—Approximately 14 weeks.
   Container size of the plant described.—16 cm.
   Growth habit.—Mounded.
   Height.—Average range 25-30 cm.
   Plant spread.—40 cm.
   Growth rate.—Vigorous.
   Branching characteristics.—Highly branched.
   Length of primary lateral branches.—20 cm.
   Quantity of primary lateral branches.—5-10.
   Characteristics of primary lateral branches.—Form: Oval shape Diameter: 1 cm. Color: Near RHS Brown N199B Texture: Smooth Strength: Strong, very stable.
   Internode length.—5-8 cm.
Foliage:
   Leaf.—Arrangement: Alternate Quantity: Approximately 20-25 per branch. Average Length: 10 cm. Average Width: 3.5 cm. Shape of blade: Lanceolate Apex: Apiculate Base: Acuminate Margin: Ciliate Texture of top surface: Smooth Pubescence: None Aspect: 45 degrees Color: Young foliage upper side: Near RHS Green 137A Young foliage under side: Near RHS Green 138B Mature foliage upper side: Near RHS Green N137A Mature foliage under side: Near RHS Green 147C Venation: Type: Pinnate. Venation color upper side: Near RHS Greyed-green 194C Venation color under side: Near RHS Red-purple 60C Petiole: Length: 1.5 cm. Diameter: 0.5 cm. Color: Near RHS Greyed-purple 184C Texture: Smooth.
Flower:
   Natural flowering season.—Flowers at any day length. All year round in a controlled greenhouse environment. Flowering is continuous from spring until fall in an outdoor garden.
   Days to flowering from rooted cutting.—10 weeks.
   Inflorescence and flower type and habit.—Single flowers raised above the foliage. The corolla consists of five petals.
   Rate of flower opening.—10 to 15 days from bud to fully opened flower.
   Flower longevity on plant.—5-8 days.
   Persistent or self-cleaning.—Self-cleaning.
   Bud.—Shape: Ovate Length: 2 cm Diameter: 1.5 cm Color: Near RHS Red 46D.
   Flower size.—Diameter: 8 cm. Depth: Approximately 7.0 including spur.
   Petals.—Quantity: 5 Arrangement: Overlapping Length: 4.5 cm Width: 4 cm Shape: Obcordate Apex: Cordate. Base: Acute Margin: Entire Texture, upper and lower surfaces: Smooth Color: When opening, upper surface: Near RHS Red 42A When opening, lower surface: Near RHS Red 41 B Fully opened, upper surface: Near RHS Red 44A Fully opened, lower surface: Near RHS Red 41 C.
   Spurs.—Quantity per flower: 1 Length: 7 cm Diameter: 0.2 cm Aspect: Spur curvature 40°-45° from peduncle Color: Towards base: Near RHS Red 47A Apex: Near RHS Red 53A.
   Sepals.—Quantity: 2 Length: 1.5 cm Width: 0.8 cm Shape (overall): Oblong Apex: Caudate Base: Rounded Margin: Entire Color: Near RHS Yellow-green 147C, upper and lower surfaces.
   Peduncles.—Length: 6 cm Diameter: 0.3 cm Angle: About 45° to the lateral branch Strength: Stable, flexible Texture: Smooth Color: Near RHS Greyed-purple 181 B.
   Fragrance.—None.
Reproductive organs:
   Stamens.—There are five stamens with anthers that are more or less fused and form a cap over the ovary. Number: 5.
   Anthers.—Shape: Oblong Length: Approximately 0.5cm. Color: Near RHS Red 41B Pollen: Color: Near RHS White 155D Quantity: Few.
   Pistil.—Number: 1 Length: 0.5 cm. Style: Length: 0.5 cm. Color: Near RHS Yellow-green 144B Stigma: Shape: Round Color: Near RHS White 155D Ovary Color: Near RHS Yellow-green 144D.
Other characteristics:
   Seeds and fruits.—5-10 seeds per fruit. Minute, dust-like, brown-grey in color. Color not accurately measured with RHS chart due to size.
   Disease/pest resistance.—Neither resistance nor susceptibility to normal diseases and pests of Impatiens have been observed.
   Temperature tolerance.—Tolerant to temperatures between 5° C. and 30° C. (grows best at 10° C.-28° C.)

What is claimed is:

1. A new and distinct cultivar of Impatiens plant named ‘DHARSSCRLT’ as herein illustrated and described.
